(19) United States
(12) Patent Application Publication
(10) Pub. No.: US 2008/0262223 A1
(43) Pub. Date: Oct. 23, 2008

(54) Isolation of Galanthamine From Biological Material

(75) Inventors: Ladislav Cvak, Opava (CZ); Martin Buchta, Ludgerovice (CZ); Jiri Faustmann, Opava (CZ); Pavel Stverka, Velka Polom (CZ); Alexandr Jegorov, Dobra Voda (CZ)

Correspondence Address: LERNER, DAVID, LITTENBERG,;KRUMHOLZ & MENTLIK, 600 SOUTH AVENUE WEST, WESTFIELD, NJ, 07090, US

(73) Assignee: IVAX Pharmaceuticals s.r.o., Opava 9 (CZ)

(21) Appl. No.: 11/596,758

(22) PCT Filed: Mar. 17, 2006
(86) PCT No.: PCT/US06/10247

§ 371 Date: Jun. 24, 2008

Related U.S. Application Data
(60) Provisional application No. 60/662,585, filed on Mar. 17, 2005.

(51) Int. Cl. C07D 223/16

(52) U.S. Cl. 540/581

(57) ABSTRACT

The subject matter of present invention relates to the process for isolation and purification of galanthamine and its derivatives produced by numerous plants.

### FIELD OF THE INVENTION

This invention relates to the process of isolating galanthamine and its derivatives in substantially pure form which overcomes the drawbacks of known processes.

### BACKGROUND OF THE INVENTION

Galanthamine, 4a,5,9,10,11,12-hexahydro-3-methoxy-11-methyl-6H-benzofuro[3a,3,2-ef]-(2)-benzazepine-6-ol, is a natural alkaloid produced by plants of the family Amaryllidaceae, e.g., genus of Galanthus, Narcissus, Leucojum and Lycoris. Its structure is depicted in FIG. 1. It was first isolated by Proskurnina and Jakovleva from Galanthus woronowi (J. Gen. Chem. USSR, 1952, 22, 1899-1902).

Pharmacologically, galanthamine is a reversible cholinesterase inhibitor like physostigmine but it is substantially less toxic. It also has analgesic and antioxidant properties. This unique combination of properties enables its use for treatment of Alzheimer disease (e.g., L. J. Scott and K. L. Goa, Drugs 2000, 60, 1095-1122) and also alcohol, drugs and nicotine addiction and some other diseases (U.S. Pat. No. 5,643,905).

Although numerous processes for synthesis of galanthamine were described (e.g. Kametani et al., J. Chem. Soc. C, 1971, 6, 1043-1047, or Shimizu et al., Heterocycles, 1977, 8, 277-282, and numerous patents), isolation of galanthamine from plant material is still a useful alternative for large scale manufacture. The common drawback of the known processes for isolation of galanthamine from plant material is the lack of robustness and scalability for large scale isolation. They were usually tailor made for processing of one defined source of plant material. When used for processing of another plant material, they are not capable of producing enough substantially pure galanthamine. The use of toxic and/or environmentally harmful solvents like dichloroethane and other chlorinated hydrocarbons and diethyl ether also discourages these processes for large scale production. Also, some operations employed in these known processes are difficult to scale up, e.g., concentration of the primary extract to dryness and dissolution of the residue in another solvent.

The patent. DE 1,193,061 describes isolation of galanthamine from plants of Amaryllidaceae by extraction of plant material alkalized with aqueous ammonia with dichloroethane or other chlorinated hydrocarbons (dichloromethane, chloroform). The obtained primary extract is further treated with diluted sulfuric acid and the accompanying alkaloids are precipitated with aqueous ammonia. Galanthamine remaining in the solution is extracted with diethylether or dichloromethane and further purified. A substantial improvement of this process is provided in the U.S. Pat. No. 5,877,172. The comminuted plant material (Narcissus pseudonarcissus “Carlton”) is prior to the extraction mixed with powdered sodium carbonate and then extracted with dichloroethane. Further processing of the primary extract is similar as described above but the formation of emulsions is minimized. Nevertheless, the used solvents, dichloroethane and diethyl ether, are not suitable for industrial scale isolation.

The U.S. Pat. No. 5,877,172 also describes extraction of the plant material alkalized prior to the extraction by addition of powdered sodium carbonate with gasoline to obtain primary extract. The primary extract is evaporated to dryness and the dry residue is dissolved in diluted sulfuric acid wherein pH of the solution is adjusted to about 4 and accompanied components of non alkaloid character are extracted by diethyl ether. The obtained refined aqueous solution is alkalized to pH of 9 and the alkaloids are extracted into diethyl ether. The diethyl ether extract is concentrated to dryness followed by the crystallization from 2-propanol to yield galanthamine. Although the use of toxic dichloroethane was eliminated, the process still uses diethyl ether. Moreover the extraction with gasoline is ineffective and requires high volume of solvent. Also, the evaporation of the primary extract to the dry residue is not convenient. The process includes several operations for separation of alkaloids from the ballast, non alkaloid components, but only operation, which assures separation of galanthamine from the other alkaloids, is the crystallization of the alkaloid concentrate from 2-propanol. This fact means that the process is not robust enough to assure the isolation of pure galanthamine from such complex material as described below.

The outline of the state of the art of the extraction of galanthamine from the plant material gives evidence that a robust process for large scale extraction and purification of galanthamine affording high pure product from different plant material is still desirable.

### BRIEF DESCRIPTION OF THE DRAWINGS

FIG. 1. Structural formula of galanthamine

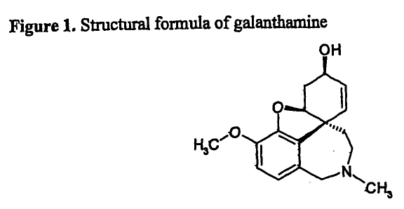

FIG. 2. HPLC analysis of the primary extract obtained in the Example 1.

FIG. 3. HPLC analysis of the crude alkaloid concentrate obtained in the Example 1.

FIG. 4. HPLC analysis of the purified galanthamine obtained in the Example 1.

FIG. 5. HPLC analysis of the galanthamine hydrochloride obtained in the Example 1.

FIG. 6. HPLC analysis of the base of galanthamine obtained in the Example 1.

FIG. 7. HPLC analysis of the base of galanthamine obtained in the Example 2.

FIG. 8. HPLC analysis of the primary extract obtained in the Example 3.

FIG. 9. HPLC analysis of the base of galanthamine obtained in the Example 3.

### SUMMARY OF THE INVENTION

In one aspect, the present invention provides a robust and efficient process for large scale isolation of galanthamine from all known plants producing galanthamine, that are the plants of Amaryllidaceae family, e.g., plants of genera of Galanthus, Narcissus, Leucojum and Lycoris. The used plant material can be dried, e.g. dried leaves or whole aerial parts of the plants, or fresh, e.g. comminuted bulbs and/or aerial parts.

In another aspect, the present invention provides a process for isolation of galanthamine comprising extraction of the plant material with aqueous solution of inorganic or organic acid, thus obtaining a primary extract and adsorption of organic compounds from the primary extract on an adsorbent, washing the adsorbent with water and elution of the organic compounds from the adsorbent with a water miscible organic solvent, thus obtaining the concentrate of alkaloids.

In another aspect, the present invention provides a process for further purification of the concentrate of alkaloids comprising adsorption of alkaloids from the concentrate of alkaloids on a cation exchange polymer resin and elution of alkaloids from the resin with aqueous solution of an inorganic base, obtaining an aqueous alkaloid concentrate.

In another aspect, the present invention provides a process for further purification of the aqueous alkaloid concentrate comprising extraction of alkaloids from the aqueous alkaloid concentrate into an organic solvent not miscible with water and concentrating of the extract obtaining a crude alkaloid concentrate.

In another aspect, the present invention provides a process for further purification of the crude alkaloid concentrate comprising a chromatographic purification of the crude alkaloid concentrate on alumina obtaining a galanthamine fraction using an organic solvent not miscible with water as mobile phase obtaining a purified galanthamine.

In another aspect, the present invention provides a process for further purification of galanthamine comprising crystallization of the purified galanthamine from a suitable solvent, obtaining purified crystalline galanthamine.

In another aspect, the present invention provides a process for further purification of galanthamine comprising re-crystallization of crystalline galanthamine from methyl isobutyl ketone or tert-butyl methyl ether.

In another aspect, the present invention provides a process for further purification of galanthamine comprising liberating galanthamine base from the galanthamine hydrochloride and its crystallization from methyl isobutyl ketone or tert-butyl methyl ether.

In another aspect, the present invention provides a means for removal of narwedine by its reduction with a suitable reducing agent capable of reducing the carbonyl group of narwedine to the secondary alcoholic group.

In another aspect, the present invention provides a process for isolation of high pure galanthamine from all the above mentioned types of plant material. The purity of the isolated galanthamine is more than 80%, preferably more than 90% and even more preferably more than 99%.

In another aspect, the invention provides a process for isolation of substantially pure galanthamine without the use of highly toxic solvents or solvents harmful for environment, e.g., chlorinated hydrocarbons.

### DETAILED DESCRIPTION OF THE INVENTION

The present invention provides a process for isolation of high pure galanthamine from a biomass. The term biomass means dried or fresh parts of plants producing galanthamine, i.e., plants of Amaryllidaceae family, e.g., plants of genera Galanthus, Narcissus, Leucojum, and Lycoris. The process consists of several consecutive steps, which were designed to optimize the efficiency of the isolation process and the capability to remove the undesirable alkaloids which are present in the plant material and must be regarded as the potential impurities of galanthamine. The individual steps were designed in order to avoid the use of toxic solvents or solvents harmful for the environment like chlorinated hydrocarbons and low boiling solvents like diethyl ether, acetone and petroleum ether. The order of individual steps was designed to make overall process efficient.

The first step of the isolation process is extraction of the biomass. It was found by experimentation that diluted aqueous solution of inorganic or organic acids are excellent means for extraction of the plant material. In contrast to known procedures using organic solvents, the aqueous extraction has several advantages: 1) it is not necessary to alkalize the plant material prior to the extraction which eliminates one mechanical operation from the process (mixing of the plant material with powdered sodium carbonate as described in the U.S. Pat. No. 5,877,172); 2) impact on environment is minimized when the extraction solvent is water; 3) it is not necessary to remove the solvent from the extracted material which eliminates another operation from the process (drying of the exhausted biomass); 4) the recovery process of the used solvent is eliminated as well. The selection of the acid used is not critical from the extraction efficiency and selectivity aspects. The selection of the acid is determined by its price, handling and its impact on corrosion of the equipment used if any. The use of phosphoric acid in concentration about 0.1% (w/w) is beneficial. Another advantage of the use of aqueous extraction of the biomass is evident when fresh plant material, e.g., bulbs, are extracted. Since such material contains a lot of water, its extraction with an organic solvent, moreover not miscible with water, will be counterproductive.

The extraction of the biomass can be accomplished by different ways, nevertheless, the use of a battery of percolators is very convenient. The use of battery can minimize the volume of the primary extract. The Example 1 documents that only about 1.5 l of primary extract was obtained from 1 kg of bulbs Nevertheless, when dried biomass is subjected to the extraction on a battery of percolators, the volume of the primary extract is substantially larger due to the low bulk density of the extracted material and large dead volume in the percolator. On the other hand, the dried material need not be comminuted when it is extracted on a battery of percolators as documented in Example 3, where a quantitative extraction was reached using the extraction ratio between the primary extract and dried plant material of 15:1 (v/w).

Next operation of the isolation process is the adsorption of organic compounds present in the primary extract on an adsorbent. It was found out by experimentation that non ionic polymer resin is very suitable adsorbent for this purpose. It was also found out by experimentation that the alkaloids present in the primary extract in form of salts must by transferred to a base form by addition of some inorganic base, e.g., sodium or potassium hydroxide. Then the alkaloids are very efficiently adsorbed on the resin together with some other organic compounds of medium polarity present in the primary extract. Polar organic compounds like saccharides and inorganic compounds are not adsorbed and thus the operation represents an important purification step. The polymeric resin suitable for the adsorption is any poly(styrene-divinylbenzene) co-polymer. The particle size distribution and pore size of the resin are not critical parameters for the efficiency or for the selectivity of the adsorption. The adsorption could be accomplished, e.g., by mixing of the resin with alkalized primary extract, nevertheless, the most convenient is the adsorption on the resin filled in a column. Then the alkalized primary extract can be simply loaded on the column, followed by washing the column with water, and elution of alkaloids and other organic compounds by an organic solvent miscible with water, or a mixture of such solvent and water, conveniently by 60% (v/v) aqueous ethanol, obtaining a concentrate of alkaloids. The concentrate contains all the alkaloids present in the primary extract and some other organic compounds.

The concentrate of alkaloids is further subjected to the adsorption of alkaloids on a cation exchange polymer resin. It was found, that the concentrate of alkaloids obtained by elution from the non ionic polymer resin can be directly loaded on a column filled with the cation exchanger. Although several types of cation exchangers were successfully used, the best results were obtained using strongly acidic cation exchange resin of gel type that is a polysulphonated (styrene-divinylbenzene) co-polymer, crosslinked with not more than 4% of divinylbenzene. Using such type of cation exchange resin, the alkaloids are quantitatively retained while the other organic compounds present in the concentrate are removed. The alkaloids are then desorbed from the column by the elution with aqueous solution of a suitable inorganic base, conveniently, by diluted aqueous ammonia, obtaining thus an aqueous alkaloid concentrate.

The adsorption of alkaloids on a cation-exchanger can be omitted in the case when the content of the organic compounds other than alkaloids in the concentrate of organic compounds is low. Such case is demonstrated in Example 3. Then the concentrate of alkaloids obtained by elution from the non ionic polymer resin can be concentrated to remove the organic solvent and the residual aqueous solution can be alkalized by addition of aqueous ammonia and aqueous alkaloid concentrate obtained by such a way can be subjected to the next purification in a similar way, as the aqueous concentrate obtained by elution from the cation exchanger.

Galanthamine and other lipophilic alkaloids are further extracted from the aqueous solution into an organic solvent. The extract is then concentrated and the obtained crude alkaloid concentrate is further purified by chromatography on alumina. Any solvent not miscible with water with exception of aliphatic hydrocarbons can be used for the extraction of alkaloids from the aqueous concentrate, but the preferred solvents are toluene, methyl isobutyl ketone and/or some esters of acetic acid e.g. propyl acetate, isopropyl acetate, butyl acetate and isobutyl acetate. The advantage of their use is based on fact that identical solvent can be used for chromatographic purification of the crude alkaloid concentrate on alumina so that the mixing of different solvents is minimized and solvent recovery is very simple.

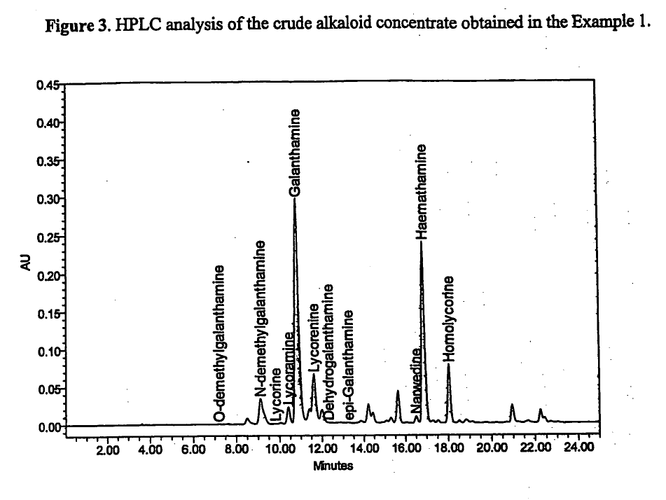

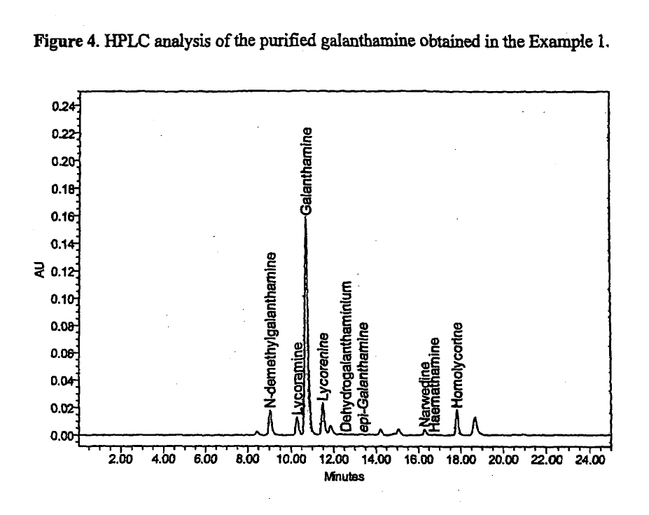

The chromatographic purification of the crude alkaloid concentrate is the first operation capable of separating individual alkaloid. Especially heamanthamine present mainly in the concentrate obtained from plants of Narcissus genus is efficiently separated by this operation as demonstrated in the Example 1 (compare FIGS. 3 and 4). The above mentioned solvents enable to perform the chromatographic separation of galanthamine from heamanthamine and some other alkaloids in isocratic mode which is very convenient for large scale preparation. While galanthamine is eluted from the chromatographic column as the main fraction (the purified galanthamine), some other alkaloids, mainly heamanthamine are captured on the column. The composition of the purified galanthamine is such that relatively pure galanthamine can be obtained in exceptionally high yield by its crystallization. The crystallization of galanthamine from the purified galanthamine can be achieved by two ways: as a salt with hydrochloric acid, or as a base. While the crystallization of the salts of galanthamine gives very high yield of the crystalline product, its impact on product purity is only moderate. It was surprisingly found, that base of galanthamine crystallizes from some solvents not described in the literature, e.g., from methyl isobutyl ketone or tert-butyl methyl ether. Crystallization from these solvents very efficiently eliminates most of the potential impurities. Very efficient is the combination of both possibilities: crystallization of galanthamine hydrochloride and then liberating galanthamine base and its crystallization as described in Example 1. Although two crystallization steps are involved in the process, the cumulative yield is surprisingly high and the combination of two different crystallization steps yields galanthamine with purity higher than 99%.

The combination of chromatography on alumina and crystallization makes it possible to eliminate most of potential impurities with the exception of N-demethylgalanthamine and narwedine as demonstrated in Examples 1 and 3. Narwedine, the biosynthetic precursor of galanthamine, is always present in the plant material used for the isolation of galanthamine. It is practically not eliminated by such a simple chromatography on alumina as described above, and only partially eliminated by crystallization. Another disclosure of the present invention makes it possible to eliminate narwedine from galanthamine. It was found that crystalline galanthamine containing more than about 0.5% narwedine can be purified by reduction of narwedine using a reducing agent capable to reduce the carbonyl group of narwedine providing thus a secondary alcoholic group of galanthamine or epigalanthamine. Such reduction can be accomplished by numerous reducing agents, but exceptionally convenient is the use of sodium borohydride. The galanthamine hydrochloride containing narwedine is dissolved in water and small amount of sodium borohydride is added. Galanthamine isolated from such reaction mixture practically does not contain any narwedine as demonstrated in Example 2.

The process according to the invention is capable of isolating galanthamine of high purity from all above mentioned plant materials. The purity of the product depends on the used plant material, but it was never less than 99%, and in some cases, the purity of isolated galanthamine was even more than 99.5%. Also the yield of the process was very high, usually more than 80% of the calculated amount as demonstrated in Examples 1 and 3.

The present invention is described in the examples below:

### EXAMPLES

The following examples illustrate but do not limit the invention.

### Example 1

### Isolation of Galanthamine Hydrochloride from the Bulbs of Narcissus pseudonarcissus “Carlton”

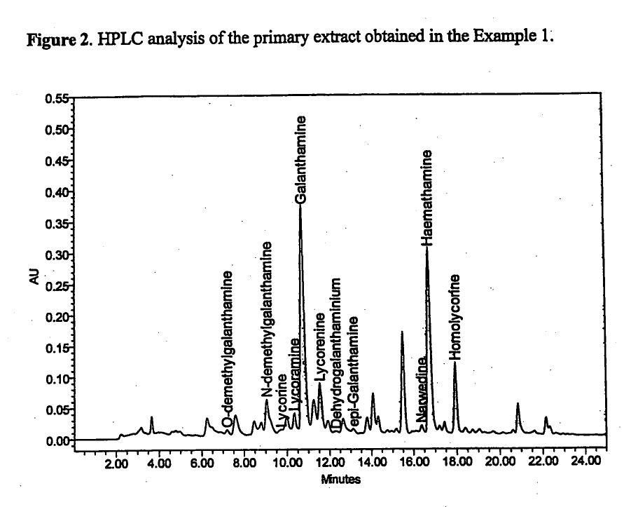

Bulbs of narcissus (Narcissus pseudonarcissus “Carlton”) containing 0.12% of galanthamine (determined by HPLC) were comminuted and filled into pilot plant battery of percolators 4×100 l (75 kg of comminuted bulbs was filled into one extractor). Individual filled extractors were joined to the battery and extracted with 0.1% (w/w) aqueous solution of phosphoric acid counter current way. 125 l of primary extract was obtained from one extractor. The HPLC record of the analysis of the primary extract is presented on FIG. 2. The primary extract from one extractor was alkalized with 10% aqueous solution of potassium hydroxide to pH 9-10 and the solution was loaded on a 60 l column filled with non-ionic resin SP-825L. The column was further washed with 100 l water and the organic compounds were desorbed from the resin by elution with 60% (v/v) aqueous ethanol, obtaining 220 l of the concentrate of alkaloids. The concentrate was further loaded on a 3 l column containing cation exchange resin SK 104, where all alkaloids were adsorbed. The column was washed with water and alkaloids were eluted from the column with 0.5% (w/w) aqueous ammonia, obtaining 30 l of aqueous alkaloid concentrate.

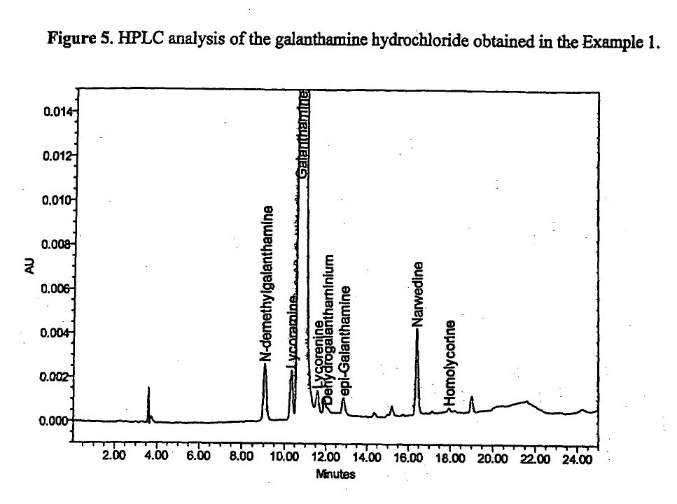

The aqueous alkaloid concentrate was extracted with 30 l of methyl isobutyl ketone and the extract was evaporated obtaining about 1 liter of crude alkaloid concentrate. According to HPLC analysis, the concentrate contained 45.8% of galanthamine in dryness—the HPLC record is presented on FIG. 3. The concentrate was subjected to chromatography on a column containing 2 kg of basic alumina, using methyl isobutyl ketone as a mobile phase. Fractions containing galanthamine (TLC monitoring) were pooled and concentrated, obtaining 134 g of dry residue (purified galanthamine). Its HPLC analysis is presented on FIG. 4. The purified galanthamine was dissolved in 450 ml of ethanol and the pH of the solution was adjusted to about 4 by addition of concentrated hydrochloric acid. The suspension of crystalline galanthamine hydrochloride was cooled in refrigerator and then the crystalline product was filtered off, washed with 100 ml ethanol and dried, obtaining 106 g of galanthamine hydrochloride. Its HPLC analysis is presented on FIG. 5.

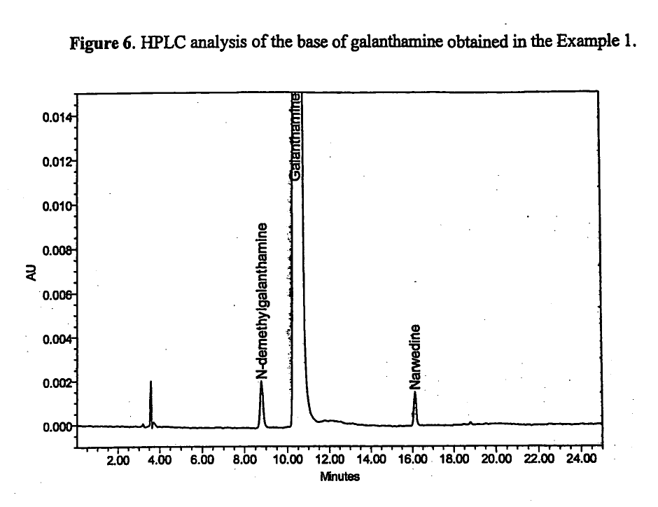

30 g of galanthamine hydrochloride prepared above was dissolved in 50 ml of hot water and 12 ml of aqueous ammonia was added to the solution. Crystalline base of galanthamine was separated by filtration and dried. Dry base of galanthamine was recrystallized from 100 ml of methyl isobutyl ketone, obtaining 21.9 g of galanthamine, which purity was determined by HPLC as 99.4%, (the HPLC record is presented on FIG. 6). The yield of the process was up to this base of galanthamine 85.0% of the theory, without recovery of the second crop from the mother liquors.

### Example 2

### Purification of Galanthamine Hydrochloride

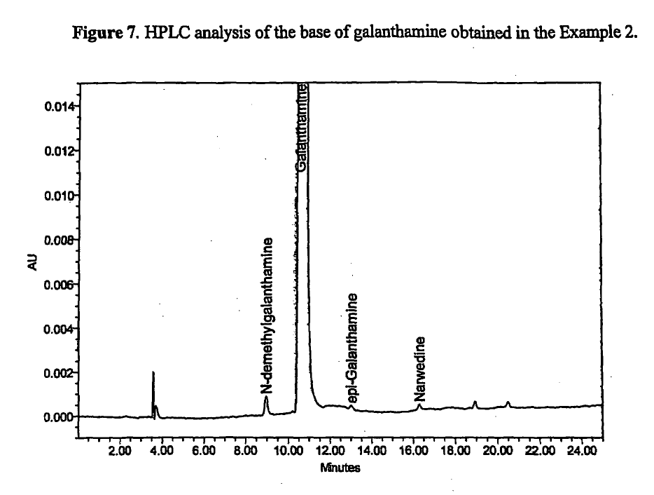

30 g of galanthamine hydrochloride prepared in Example 1 and containing according to HPLC analysis 1.1% of narwedine was dissolved in 120 ml of water and 1.2 g of sodium borohydride was added in six portions within about 30 minutes under stirring. The solution was stirred for another 30 minutes at laboratory temperature and then 12 ml of 25% (w/w) aqueous ammonia and 200 ml of methyl isobutyl ketone were added to the solution. The organic phase was separated, concentrated to the volume about 100 ml and let to crystallize in refrigerator for 24 hours. The crystalline base of galanthamine was separated by filtration and dried, obtaining 19.3 g of galanthamine, which purity was determined by HPLC as 99.7% and where the content of narwedine was 0.04% (the HPLC record is presented on FIG. 7).

### Example 3

### Isolation of Galanthamine (Base) from the Dried Leaves of Leucojum aestivum, L

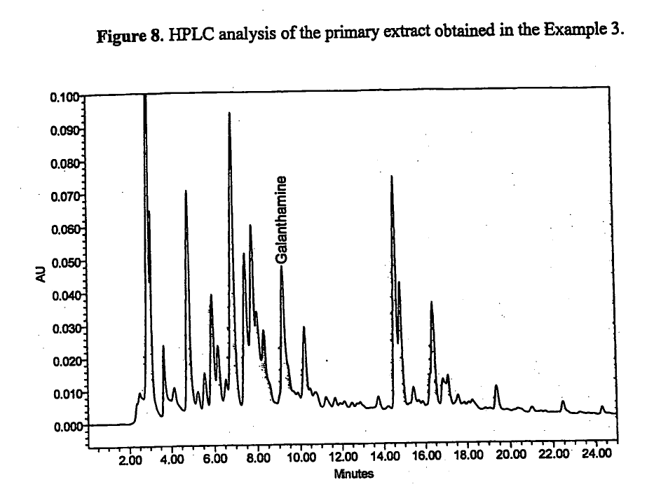

40 kg of dried leaves of snowflakes (Leucojum aestivum, L.) containing 0.26% of galanthamine (determined by HPLC) was comminuted and filled into pilot plant battery of percolators 4×100 l (10 kg of comminuted leaves was filled into one extractor). Individual filled extractors were joined to the battery and extracted with 0.1% (w/w) aqueous solution of phosphoric acid by counter-current way. 150 l of primary extract was obtained from one extractor. The HPLC record of the analysis of the primary extract is presented on FIG. 8. The primary extract from one extractor was directly adsorbed on 60 L of non-ionic resin SP-825L filled in column, washed with 100 l of water and desorbed with 90% (v/v) of aqueous ethanol (200 l) and then with 50 l of water, obtaining a concentrate of alkaloids.

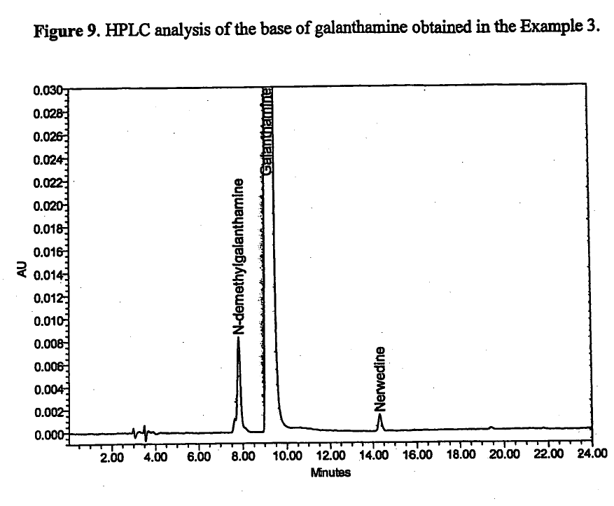

The concentrates of alkaloids obtained from all four extractors were combined and evaporated to the volume of 75 l, diluted with water to the final volume of 300 l and pH of the solution was adjusted to about 10 by addition of aqueous ammonia, obtaining aqueous alkaloid concentrate. The concentrate was extracted with 200 l of methyl isobutyl ketone in continuous counter-current extractor. Resulted extract was evaporated to volume of about 1000 ml and loaded on a column filled with 1000 g of basic alumina. The column was eluted with methyl isobutyl ketone and the fractions containing galanthamine (TLC monitoring) were pooled and evaporated to dryness, obtaining 112.5 g of residue (purified galanthamine). The residue was crystallized from 340 ml of tert-butyl methyl ether, obtaining 83.6 g of galanthamine of 99.0% purity as determined by HPLC (the HPLC record is presented on FIG. 9). The yield of the process was 80.0% of the theoretical, without recovery of the second crop from the mother liquors.

The process of current invention can be adapted for galanthamine derivatives also. While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

What is claim is:

1. A process for isolation of galanthamine from galanthamine containing biomaterial comprising:
a) extraction of the biomaterial with aqueous solution of suitable organic or inorganic acid obtaining thus a primary extract
b) adsorption of the organic compounds from the primary extract on an absorbent, washing the adsorbent with water, and elution of the organic compounds from the adsorbent using a water miscible organic solvent, obtaining a concentrate of alkaloids.

2. The process according to the claim 1, wherein the galanthamine containing biomaterial are dried or fresh parts of plants of Amaryllidaceae family.

3. The process according to the claim 2, wherein the plants of Amaryllidaceae family are plants of the genera Galanthus, Narcissus, Leucojum and/or Lycoris.

4. The process according to the claim 2, wherein the fresh parts of plants are bulbs and or whole aerial parts of the plants.

5. The process according to the claim 2, wherein the dried parts are leaves or the whole aerial parts of the plants.

6. The process according to the claim 1, wherein the organic or inorganic acid used for the extraction of the biomass is selected from group consisting of acetic acid, tartaric acid, citric acid, phosphoric acid, sulfuric acid and hydrochloric acid.

7. The process according to the claim 6, wherein the concentration of the organic or inorganic acid in water is from 0.05 to about 2%.

8. The process according to the claim 1, wherein the biomass is extracted with aqueous solution of phosphoric acid in concentration about 0.1% (w/w).

9. The process according to the claim 1, wherein the extraction of the biomass is accomplished counter current way on a battery of percolators.

10. The process according to the claim 1, wherein from 1 to 3 weight parts of the primary extract are obtained from one weight part of the biomass.

11. The process according to the claim 1, wherein the adsorbent used for the adsorption of alkaloids from the primary extract is a poly(styrene-divinylbenzene) co-polymer.

12. The process according to the claim 1, wherein the adsorption of alkaloids on the adsorbent is accomplished at the pH from about 8 to about 11.

13. The process according to the claim 1, wherein the polar organic solvent used for the elution of alkaloids from the adsorbent is methanol, ethanol, 2-propanol, acetone, mixtures thereof or their aqueous mixtures.

14. The process according to the claim 1, wherein the concentrate of alkaloids is further purified by adsorption of alkaloids from the concentrate on a cation exchange resin and elution of the alkaloids from the cation exchange resin is carried out with the aqueous solution of a suitable base to obtain an aqueous alkaloid concentrate.

15. The process according to the claim 14, wherein the cation exchange resin is a strongly acidic cation exchange resin of gel type.

16. The process according to the claim 14, wherein the cation exchange resin is a polysulphonated (styrene-divinylbenzene) co-polymer crosslinked with not more than 4% of divinylbenzene.

17. The process according to the claim 14, wherein the base used for the elution of alkaloids from the cation exchange resin is aqueous ammonia.

18. The process according to the claim 1, wherein the concentrate of alkaloids is concentrated in order to remove the organic solvent, obtaining thus an aqueous alkaloid concentrate.

19. The process according to the claims 14 and 18, wherein the aqueous alkaloid concentrate is further purified by extraction of the alkaloids into an organic solvent not miscible with water and the obtained extract is concentrated to obtain a crude alkaloid mixture.

20. The process according to the claim 19, wherein the organic solvent not miscible with water is methyl isobutyl ketone, propyl acetate, isopropyl acetate, butyl acetate, isobutyl acetate, toluene or mixtures thereof.

21. The process according to the claim 19, wherein the crude alkaloid mixture is further purified by a chromatography on alumina using an organic solvent not miscible with water as the mobile phase, obtaining thus a purified galanthamine.

22. The process according to the claim 21, wherein the organic solvent not miscible with water is methyl isobutyl ketone, propyl acetate, isopropyl acetate, butyl acetate, isobutyl acetate, toluene or mixtures thereof.

23. The process according to the claims 19 and 21, wherein the same solvent is used for the extraction of alkaloids from the aqueous concentrate and for the chromatographic purification.

24. The process according to the claims 19 and 21, wherein methyl isobutyl ketone is used both for extraction and chromatography.

25. The process according to the claim 21, wherein the purified galanthamine is concentrated and crystallized from methyl isobutyl ketone, acetone, or tert-butyl methyl ether, obtaining crystalline galanthamine.

26. The process according to the claim 21, wherein the purified galanthamine is concentrated, the residue is dissolved in ethanol and one equivalent of hydrochloric acid is added to facilitate galanthamine hydrochloride crystallization.

27. The process according to the claim 25, wherein the crystalline galanthamine is further purified by re-crystallization from methyl isobutyl ketone, tert-butyl methyl ether or mixtures thereof.

28. The process according to the claim 26, wherein the galanthamine hydrochloride is transferred to the base and the base is further purified by crystallization from methyl isobutyl ketone, tert-butyl methyl ether or mixtures thereof.

29. The process according to the claim 26, wherein the crystalline galanthamine hydrochloride containing more than 0.5% (w/w) of narwedine is purified by dissolving in a suitable solvent and adding a reducing agent capable of reducing the carbonyl group of narwedine to the alcoholic group.

30. The process according to the claim 29, wherein the suitable solvent is water.

31. The process according to the claim 29, wherein the reducing agent is sodium borohydride.

32. The process according to the claim 29, wherein not more than 0.1 mol of sodium borohydride is used for 1 mol of galanthamine containing more than 0.5% (w/w) narwedine.

33. The process according to the claims 27 and 28, wherein the purity of the crystalline galanthamine obtained is more than 99%.

34. The process according to the claims 27 and 28, wherein the crystalline galanthamine is used for preparation of galanthamine hydrobromide.

35. A process for isolation of galanthamine from galanthamine containing biomaterial comprising:
a) extraction of the biomaterial with aqueous solution of suitable organic or inorganic acid obtaining thus a primary extract
b) adsorption of the organic compounds from the primary extract on an absorbent,
c) elution of the organic compounds from the adsorbent to obtain a concentrate of alkaloids.

36. The process according to the claim 35 characterized in that the obtained galanthamine is recrystallized from a suitable medium.

37. The process according to claim 35 wherein galanthamine obtained is used in pharmaceutical dosage forms.
